(19) United States
(12) Patent Application Publication
(10) Pub. No.: US 2007/0191805 A1
(43) Pub. Date: Aug. 16, 2007

(54) Tampon, in particular for femine hygiene

(75) Inventor: Robert Kramer, Koln (DE)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: JOHNSON & JOHNSON GMBH, Dusseldorf (DE)

(21) Appl. No.: 10/553,020

(22) PCT Filed: Apr. 13, 2004
(86) PCT No.: PCT/EP04/03871

§ 371 Date: Dec. 19, 2006

(30) Foreign Application Priority Data
Apr. 9, 2003 (DE) 103 16 234.8

(51) Int. Cl. A61F 13/20

(52) U.S. Cl. 604/385.18; 604/904

(57) ABSTRACT

The tampon according to the invention comprises a tampon body and a withdrawal tape fastened on the tampon body. The withdrawal tape is configured such that its effective length can be changed preferably before use, but also during use. In particular, the withdrawal tape is formed with at least one loop or a fold which reduces the effective length of the withdrawal tape and can be released in order to increase the effective length of the withdrawal tape. Effective length refers to the extent of the withdrawal tape, measured from the rear end of the tampon body, which is available for the user to grip.

The invention relates to a tampon, in particular for feminine hygiene, which has a tampon body and a withdrawal tape fastened on the tampon body.

A tampon, in particular a tampon for feminine hygiene, is provided with a withdrawal tape in order for it to be possible for the used tampon easily to be pulled out of, and removed from, the user's vaginal opening. The withdrawal tape is anchored firmly in the tampon body and usually comprises one or more threads which have a length of approximately 10 to 15 cm, measured from the rear end of the tampon body. In a number of configurations, the withdrawal tape comprises two withdrawal threads which are knotted together at their end.

It has been found, in practice, that in particular young women who play sports want a tampon with a shorter and as far as possible “invisible” withdrawal tape. There are various reasons for this.

While urinating, it would be extremely difficult and impractical to grip the withdrawal tape in order to keep it away from the flow of urine, so that most women accept that the withdrawal tape will become wet and saturated with liquid when they urinate. This may both render the tampon uncomfortable to wear, as a result of the damp withdrawal tape, and be a source of infection.

A recent test, furthermore, has established that, during menstruation, women choose to use tampons for sport and during leisure time and holidays because tampons afford largely invisible protection, which was one of the main concerns of the users. For example more than 50% of those taking part in the tests gave this as a desirable effect of tampons used as protection during menstruation. It has also been found that a large number of users find the visibility of the withdrawal tape in swimming pools and saunas and on the beach both unpleasant and embarrassing. On account of its customary length of approximately 10 to 15 cm, measured from the rear end of the tampon, a withdrawal tape may be visible when the tampon is worn, e.g. for swimming, in the sauna, etc.

There is thus a need for tampons for feminine hygiene which have a short, as far as possible invisible withdrawal tape, in particular for use in specific situations, such as for sport, in the sauna, on the beach, etc. On the other hand, many women prefer, in most situations, tampons with a withdrawal tape of customary length, because this is easier to grip and to handle in order to pull the tampon out of the vaginal opening.

U.S. Pat. No. 6,312,419 deals with the theme of short and extensible withdrawal tapes for tampons, the problem on which this patent is based being that the standard length of the withdrawal tapes for tampons is considered too short and thus a lengthening device which can be fitted on the withdrawal tape is proposed for the better handling of the withdrawal tape.

U.S. Pat. No. 6,142,489 deals with the problem of the possibility of a withdrawal tape being saturated with liquid when one urinates, and proposes the solution of coating the withdrawal tape. This specification, however, does not deal with the length of the withdrawal tape.

The object of the invention is to specify a tampon, in particular for feminine hygiene, which allows a user to make a selection between a shorter withdrawal tape and a longer withdrawal tape. This object is achieved by a tampon having the feature of claim 1. The invention also proposes methods of producing such tampons.

The tampon according to the invention comprises a tampon body and a withdrawal tape fastened on the tampon body. The withdrawal tape is configured such that its effective length can be changed preferably before use, but also during use. In particular, the withdrawal tape is formed with at least one loop or a fold which reduces the effective length of the withdrawal tape and can be released in order to increase the effective length of the withdrawal tape. Effective length refers to the extent of the withdrawal tape, measured from the rear end of the tampon body, which is available for the user to grip.

The tampon according to the invention can be used with the shortened withdrawal tape, that is to say with the loop or fold, which has the advantage that only a short effective withdrawal tape extends from the rear end of the tampon body, which short tape is barely visible, if at all, during use and can absorb less urine and menstrual fluid. If the free end of the withdrawal tape is pulled before use, it is possible, by virtue of the pulling action, to release the loop or fold and thus increase the effective length of the withdrawal tape, with the result that the same tampon also gives the option of a withdrawal tape of standard length, which is easier to grip and to handle when the tampon is to be removed.

The invention proposes two different preferred embodiments of the tampon. In one embodiment, the withdrawal tape has one end connected to the tampon body and its other, free end knotted to form a loop which can be released by virtue of the free end being pulled. This “knotted” embodiment has the advantage that the production equipment does not have to be changed in any way during the production of the tampon, and that all that is required is for steps to be taken in order to knot the withdrawal tape to form a loop. There are a large number of standard knots which can be used in this embodiment of the invention, it being the intention for the loop to be formed such that it releases by virtue of the free end of the withdrawal tape being pulled, but cannot be released by virtue of the loop itself being pulled.

Examples of knots for forming the loop are constituted by a loop in the manner of a half-loop or in the manner of a Windsor knot. The person skilled in the art will be able to provide other types of knot.

In the second preferred configuration of the invention, the withdrawal tape is arranged partly within the tampon body and is positioned and retained in at least one fold therein. During the production of the tampon, the withdrawal tape, rather than being led out of the tampon over the shortest route, is positioned in one or more folds parallel to, obliquely in relation to or transversely to the longitudinal extent of the tampon and is integrated and retained in the tampon body by virtue of the latter being rolled and pressed. The folds of the withdrawal tape in the tampon body are designed such that they can be released by virtue of the free end of the withdrawal tape being pulled, in order to increase the effective length of the withdrawal tape outside the tampon.

This configuration has the advantage that the equipment for producing the knot in the end of the withdrawal tape does not have to be changed in any way, although a slight modification in the production process of the tampon is necessary.

The invention is explained in more detail hereinbelow by way of the preferred embodiments and with reference to the drawings, in which:

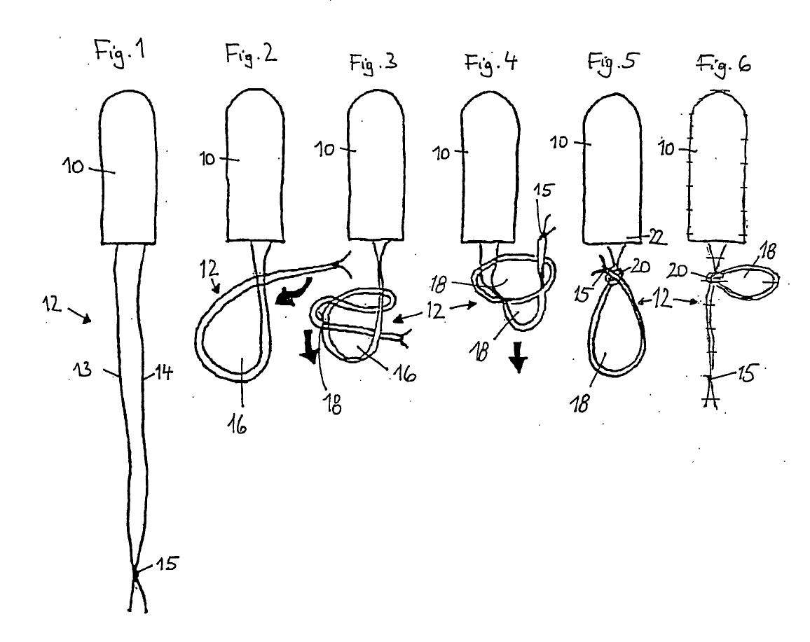

FIGS. 1 to 6 show schematic illustrations of the production and use of a tampon according to a first embodiment of the invention; and

FIGS. 7 to 10 show schematic illustrations of the steps for producing a tampon according to a second embodiment of the invention.

FIG. 1 shows a tampon comprising a tampon body 10 and a withdrawal tape 12 which is formed from two threads 13, 14 which are connected by a knot 15 at their free end.

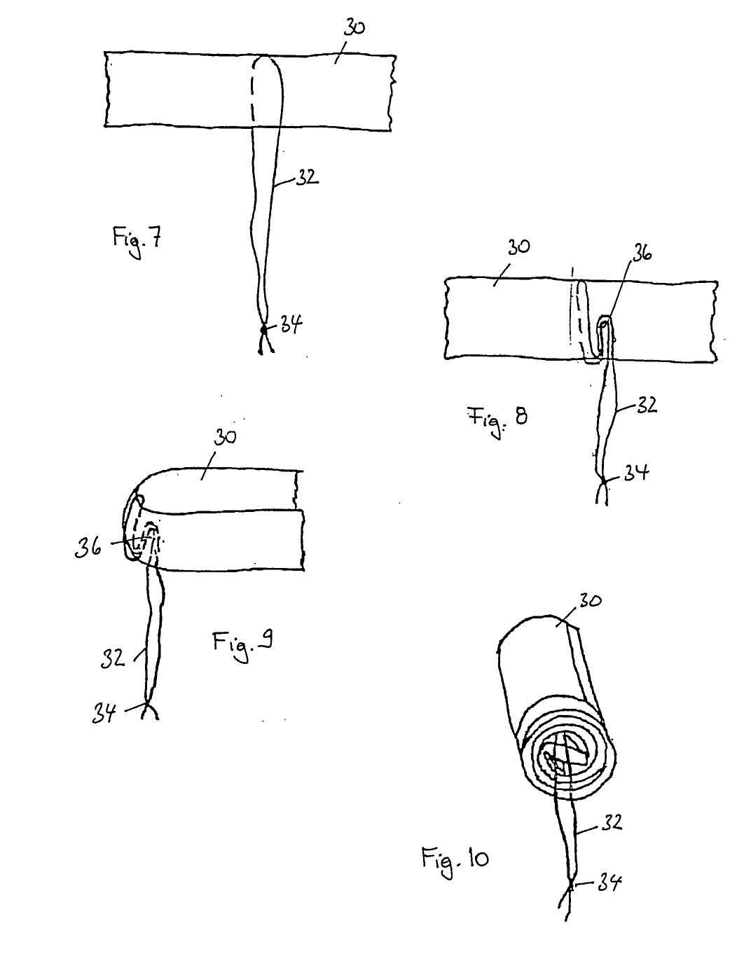

FIGS. 2 to 6 illustrate schematically how, in the first preferred embodiment of the invention, the effective length of the withdrawal tape 12 is shortened by knotting a loop and how the loop can be lengthened again. As is shown in FIG. 2, in a first step, the withdrawal tape 12 forms a loop 16. In the next step, which is illustrated in FIG. 3, a further loop 18 is formed, this being pulled through the first loop 16. In the next step, which is shown in FIG. 4, the second loop 18 is pulled away from the tampon body 10, while the first loop 16 and the knot 15 of the withdrawal tape 12 are simultaneously retained firmly in order to form the relatively large loop 18 which is shown in FIG. 5, and is retained in the vicinity of the rear end 22 of the tampon body 16 by a knot 20 formed by the first loop 16.

Forming the loop 18 produces a reduced effective length of the withdrawal tape 12, measured from the rear end 22 of the tampon body 10, this length being approximately equal to half the length of the straightened-out withdrawal tape 12 from FIG. 1.

It is easily possible, by virtue of the free end of the withdrawal tape 12 being pulled, at the knot 15, for the loop 18 to be released again in order to bring the withdrawal tape 12 back to its maximum length.

The first embodiment of the tampon according to the invention thus specifies a solution for a variable-length withdrawal tape which is particularly straightforward to realize and easy to handle. It is immediately obvious to the user that the knotted loop 18 can be used as a short withdrawal tape or alternatively can be released in order to form a longer withdrawal tape.

A further embodiment of the invention is described hereinbelow with reference to FIGS. 7 to 10.

FIG. 7 shows, schematically, part of a fabric tampon band 30 and a withdrawal tape 32 which is positioned around the fabric tampon band 30, transversely to the longitudinal extent thereof, and is knotted at its ends. The end knot is designated 34. As is shown in FIG. 7, the withdrawal tape 32 extends transversely to the fabric tampon strip 30 and laterally beyond the latter.

The arrangement shown in FIG. 7 corresponds to a standard arrangement of fabric tampon strip and withdrawal tape for producing tampons in accordance with which the withdrawal tape is positioned around a fabric tampon strip and the latter is then rolled and pressed in order to form the tampon.

According to the invention, the withdrawal tape 32 is positioned in at least one fold 36 before the fabric tampon band 30 is rolled and pressed, it being possible for this fold 36 to be positioned parallel to, obliquely in relation to or transversely to the transverse extent of the fabric tampon band 30. In contrast to the illustration of FIG. 8, it is also possible for the withdrawal tape 32 to be positioned in more than one fold 36.

As is shown in FIG. 9, the fabric tampon band 30 is then folded together, with the result that the withdrawal tape 32 with the fold 36 comes to rest in the fold of the folded fabric tampon band 30. In a following tampon-production step, which is shown in FIG. 10, the folded-together fabric tampon band 30, with the fold 36 therein, is rolled and pressed in order to form the finished tampon. The operations of folding the fabric tampon band 30 together, this being shown in FIG. 9, and of rolling and pressing the tampon may correspond, in principle, to the conventional method for producing tampons, with the exception that the withdrawal tape 32, rather than, being led away rectilinearly from the tampon, transversely to the longitudinal extent of the fabric tampon band 30, is positioned in at least one fold 36.

The withdrawal tape 32 may be folded, for example, such that the withdrawal tape can be lengthened from a short, effective length of approximately 5 to 8 cm, in particular 6.5 cm, outside the tampon body to an extended effective length of 10 to 15 cm, in particular approximately 13.5 cm. During use, the user can decide whether to use the tampon with the short withdrawal tape, in the state provided by the manufacturer, or whether, before using the tampon, to pull the withdrawal tape 32 out of the tampon until it reaches its extended length, in order that the tampon can be used with a longer withdrawal tape.

Tests have shown that, in the second embodiment of the invention, a force of 0.4 to 2.2 N is necessary in order to pull the withdrawal tape 32 out of the tampon body. Tampons with a weight of between 2.4 g and 2.7 g and a stability in the region of approximately 20 N to 35 N were used in these tests. These tests have also shown that the force for pulling the withdrawal tape out is dependent on the arrangement of the fold 36 on the fabric tampon band 30. With the fold 36 arranged parallel to the withdrawal tape 32, as is shown in FIG. 8, the force which was necessary in order to pull the withdrawal tape out of the tampon body was slightly lower than in the case of the withdrawal tape 32 being folded obliquely or laterally in relation to the transverse direction of the fabric band 30.

The features disclosed in the above description, in the claims and in the drawings may be significant both individually and in any desired combination in order to realize the various embodiments of the invention.

### LIST OF DESIGNATIONS

Tampon body 10
Withdrawal tape 12
Threads 13, 14
Knot(s) 15
First loop 16
Second loop 18
Knot(s) 20
Rear end 22
Fabric band of tampon 30
Withdrawal tape 32
End knot(s) 34
Fold 36

1. Tampon, in particular for feminine hygiene, comprising a tampon body and a withdrawal tape which is fastened on the tampon body and is configured such that the withdrawal tape has an effective length that can be changed before or during use, wherein the withdrawal tape is formed with at least one loop that reduces the effective length of the withdrawal tape and can be released in order to increase the effective length of the withdrawal tape and the withdrawal tape has a first end connected to the tampon body and a second end having a knot, a loop and a free end extending from the knot and the loop can be released by pulling the free end.

2. (canceled)

3. (canceled)

4. Tampon according to claim 1, wherein the loop is formed such that it does not release by virtue of the loop being pulled.

5. Tampon according to claim 1, wherein the loop is formed in the manner of a half-loop.

6. Tampon according to claim 1, wherein the loop is formed in the manner of a Windsor knot.

7. Tampon, in particular for feminine hygiene, comprising a tampon body and a withdrawal tape which is fastened on the tampon body-and is configured such that the withdrawal tape has an effective length that can be changed before or during use, wherein the withdrawal tape is formed with at least one fold that reduces the effective length of the withdrawal tape and can be released in order to increase the effective length of the withdrawal tape and the withdrawal tape is arranged at least partly within the tampon body and is positioned with the at least one fold therein, and a portion of the withdrawal tape having the at least one fold is retained between adjacent portions of the tampon body.

8. Tampon according to claim 7, wherein the tampon body is rolled and pressed to retain the portion of the withdrawal tape having the at least one fold.

9. Tampon according to claim 7, wherein at least one fold of the withdrawal tape within the tampon body is releasable by pulling the free end of the withdrawal tape in order to increase the effective length of the withdrawal tape outside the tampon.

10. Method of producing a tampon comprising the steps of: positioning the withdrawal tape around a fibrous tampon web such that the withdrawal tape extends transversely to and laterally beyond the fibrous tampon web; rolling and pressing the fibrous tampon web to form the tampon body with the withdrawal tape fastened therein; and knotting the withdrawal tape outside the tampon body to form a releasable loop.

11. Method of producing a tampon comprising the steps of: positioning the withdrawal tape is positioned around a fibrous tampon web such that the withdrawal tape extends transversely to the fibrous tampon web and a portion of the withdrawal tape extends laterally beyond the fibrous tampon web; and folding the portion of the withdrawal tape that extends beyond the fibrous tampon web partly onto the; rolling and pressing the fibrous tampon web to form a tampon body with the withdrawal tape fastened therein and to retain the folded portion of the withdrawal tape in the tampon.

12. Method according to claim 11, wherein the withdrawal tape is folded onto the fibrous tampon web in a direction selected from the group consisting of parallel to, obliquely in relation to, or perpendicularly to the transverse direction of the fibrous tampon web.
